SEQUENCE LISTING (2 sequences)

SEQ ID NO: 1 - protein, 237 aa, Homo sapiens
  exemplary human prostate-specific antigen (PSA) mature protein,precursor amino acid residues 25-261
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 2 - protein, 398 aa, Homo sapiens
  exemplary human alpha-1 antichymotrypsin (ACT) mature protein,precursor amino acid residues 26-423
NSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

(19) United States
(12) Patent Application Publication
(10) Pub. No.: US 2008/0125582 A1
(43) Pub. Date: May 29, 2008

(54) Methods and compositions for stabilizing prostate specific antigen

(75) Inventor: Kelly Millan, Lake Forest, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Bio-Rad Laboratories, Inc., Hercules, CA (US)

(21) Appl. No.: 11/581,066

(22) Filed: Oct. 13, 2006

(51) Int. Cl. C07K 19/00; C07K 1/113

(52) U.S. Cl. 530/403

(57) ABSTRACT

The present invention provides irreversibly linked stable protease-protease inhibitor conjugates, e.g., conjugates comprising α1-antichymotrypsin linked to prostate specific antigen (PSA) or trypsin-antitrypsin conjugates, methods of making such conjugates and methods of using the conjugates, e.g., as controls or calibrators for PSA detection assays or for multi-analyte controls.

### BACKGROUND OF THE INVENTION

Prostate-specific antigen (PSA) is a trypsin-like serine protease that binds to α-1 antichymotrypsin (ACT) and other serine protease inhibitors (serpins) such as α1-antitrypsin (AT), protease C inhibitor (PCI), and α-2 macroglobulin (A2M). The majority of total PSA in serum is bound to ACT. However, the PSA that is complexed to ACT is cleaved through hydrolysis under neutral to alkaline conditions. The PSA and ACT dissociate, leading to active free PSA molecules, which then bind other inhibitors, including A2M. PSA bound to A2M is not measurable by most commercial assays. The binding of PSA to A2M occurs by the cleavage of the enzyme of the peptide bond between amino acids Tyr 686 and Glu687 of the bait region of A2M resulting in a conformational change and entrapment of the PSA within the A2M. The result is that the antibodies that recognize PSA in immunoassays can no longer bind to PSA. Thus, the effect is a net loss of detectable free PSA and total PSA.

The poor stability of PSA-ACT in serum or other buffers creates a problem when monitoring free PSA and total PSA in test methods that monitor PSA as a biochemical marker for diagnosis and staging of prostate cancer. The hydrolysis of PSA-ACT can be controlled by selecting an optimal pH that is slightly acidic; however, there are many assays that require neutral to basic pH conditions that therefore cannot provide optimal results in detecting PSA levels. Many of such assays evaluate multiple analytes, or the assay conditions are unique, such that the optimum pH of the analysis can sometimes cause the destabilization of PSA into its various forms (free PSA and total PSA, etc). Thus there is a need for improved reagents and assays for monitoring PSA. This invention addresses that need.

### BRIEF SUMMARY OF THE INVENTION

The current invention is based on the discovery that a serine protease, e.g., PSA, and a serpin, e.g., anti-chyotrypsin (ACT) or anti-trypsin (AT), can be synthetically joined by a covalent linkage to provide a stable reagent, e.g., for controls, calibrators, or in reagents used for quantification of a serine protease, e.g., PSA. Synthetically joining of a serine protease to a serpin via a covalent bond may also provide protection to other analytes, enzymes, and proteins in the matrix in which PSA is found.

In the current invention, the covalent bond that joins the serine proteinase to the serpin, e.g., that joins PSA to a serpin such as ACT, is not naturally occurring, but is introduced synthetically by chemical synthesis or is introduced using recombinant DNA technology to link the two moieties. The non-naturally occurring bond is distinct from a naturally occurring bond, such as the acyl ester linkage between the active site serine of a protease and the reactive site loop (RSL) of a serpin, which has been described for various serpins/serine proteases. Thus, the invention provides a conjugate comprising a serine protease having a stable covalent linkage to a serpin. Preferably, the serine protease is prostate specific antigen (PSA) and the serpin is α1-antichymotrypsin (ACT). In the conjugates of the current invention, the protease, e.g., PSA, and serpin are linked by at least one synthetic covalent linkage or by recombinant linkage.

A serine protease other than PSA can also be linked by a synthetic or recombinant linkage to a serpin. Thus, in some embodiments, a serine protease such as human kallikrein or trypsin can be linked to a serpin such as ACT or anti-trypsin. In particular embodiments, the serine protease trypsin is synthetically or recombinantly linked to the serpin anti-trypsin.

The serine protease and serpin, e.g., PSA and ACT, can be linked by any type of stable covalent bond that is not a naturally occurring acyl ester bond. Thus, the serine protease and serpin, e.g., PSA-ACT, can be linked by an amide bond, an amine-amine linkage, a sulfhydryl linkage or any other stable covalent bond. It is understood by those in the art that the type of linkage can be selected to have a desired stability, e.g., based on the intended use of the conjugate.

In some embodiments, the serine protease-serpin, e.g., PSA-ACT, is linked using recombinant technology. Thus, a fusion protein is generated in which the two moieties are stably linked by an amide bond.

The invention additionally provides a method of coupling a serine protease to a serpin, the method comprising chemically linking the protease to the serpin using a cross-linking agent. In typical embodiments, the protease is PSA and the serpin is ACT. In other embodiments, the protease may be human kallikrein or trypsin, which is linked to a serpin such as anti-trypsin or ACT.

The methods of the invention employ known chemical linking reagents. For example, in some embodiments, the reagent is a maleimide crossing linking reagent.

In other embodiments, the invention provides a method of coupling a protease to a serpin using recombinant expression to generate a protease-serpin fusion protein. In typical embodiments, the protease is PSA and the serpin is ACT.

The invention also includes kits comprising stable covalently linked serpin-serine protease conjugates of the invention, e.g., PSA-ACT. Such kits can comprise additional components such as assay reagents and the like.

### BRIEF DESCRIPTION OF THE DRAWINGS

FIG. 1 provides a schematic showing naturally occurring non covalently bonded vs. covalently bonded ACT-PSA complexes.

### DETAILED DESCRIPTION OF THE INVENTION

### Introduction

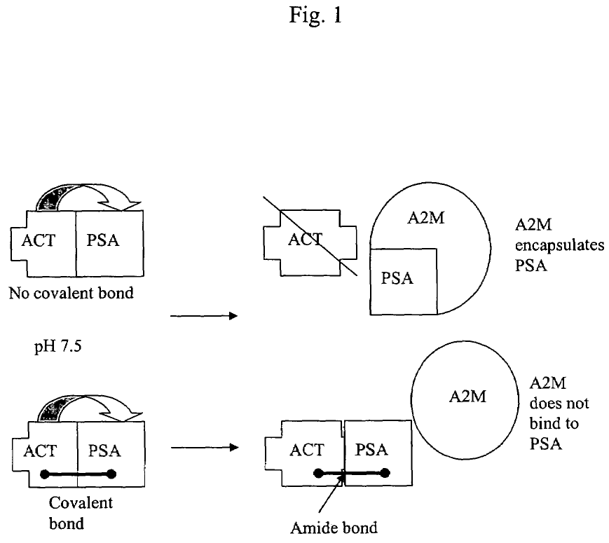

The present invention provides stable, e.g., pH-stable, serine protease-serpin conjugates, e.g., PSA-serpin conjugates, that can be used, for example, as control reagents for multianalyte analysis. The protease inhibitor-serpin conjugates are generated by synthetically or recombinantly creating at least one covalent bond that links the serpin to the proteinase. Such conjugates are stable, in contrast to ACT-PSA complexes that form spontaneously that are not stably covalently bonded (FIG. 1), but that may have an acyl ester bond that is not stable at acidic or neutral pH.

The conjugates of the invention are generated using any method known in the art, which includes both chemical linkage and recombinant techniques. Often, the coupling reaction used in the reaction produces an amide bond. Accordingly, in some embodiments, the serine protease, e.g., PSA, can be conjugated to the serpin, e.g., ACT, using a chemical coupling technique that results in at least one amide bond or using recombinant DNA technology to generate a fusion protein where the moieties are linked, directly or via a linker, by at least one amide bond.

Once the serine protease, e.g., PSA, is chemically (or recombinantly) conjugated to the serpin, the stability of the serpin and free serpin are both significantly improved in a wide variety of conditions such as in serum, in buffer and in both liquid and lyophilized states.

### Serpins

Serpins refer to a superfamily of proteins, named for the serine protease activity of many of its members. The family members are single-chain proteins, usually 40-60 kDa in size (for reviews, see for example, Bird, Results Probl Cell Differ 24:63-89 (1998); Pemberton, Cancer J 10(1):1-11 (1997); Worrall et al., Biochem Soc Trans 27(4):746-50 (1999); and Irving et al., Genome Res 10:1845-64 (2000)). Serpin family members generally share about 15-50% amino acid sequence identity. Three-dimensional computer generated models of the serpins are virtually superimposable. Serpins are found in vertebrates and animal viruses, plants and insects, and identified members of this superfamily number nearly 300. Not all serpins inhibit proteinase activity; however, in the context of this invention, the serpins used in the conjugate proteins typically have inhibitory activity. Reviewed, e.g., Silverman et al., J. Biol. Chem. 276:33293-33296, 2001. The conformation of serpins that is required for their inhibitory activity has been well documented (see, e.g., references cited in Silverman et al.).

Many serpins are found at relatively high levels in human plasma. These include ACT, AT, PCI, plasminogen activator inhibitors 1 and 2 (PAI-1 and PAI-2), tissue kallikrein inhibitor, α2-antiplasmin, and neuroserpin. These serpins have a conserved structure. Serpins typically have nine α helices and three β-pleated sheets. The reactive site loop (RSL) region contains the proteinase recognition site. The RSL is about 20 to 30 amino acids in length and is located 30 to 40 amino acids from the carboxy terminus. The RSL is exposed at the surface of the protein. The core structure of the serpin molecule folds into a three-β-sheet pear shape that presents the RSL at the top of the structure. The RSL contains so-called “bait” sequences that are believed to mimic the target proteinase's substrate and regulate the activity of specific serine proteases by mimicking the protease's substrate and covalently binding to the protease when cleaved at the RSL. When cleaved by the target protease, the serpin undergoes a conformational change that is accompanied by the insertion of the remaining reactive site loop into one of the β sheets. During this transition, serpins form a stable heat-resistant complex with the target protease. The sequence of the RSL, and in particular the P1 and adjacent amino acid residues, determine an inhibitory serpin's specificity for a protease.

Serpins have several regions involved in controlling and modulating conformational changes associated with attaching to a target protease. These are the hinge region (the P15-P9 portion of the RSL); the breach (located at top of one of the β-sheets, the A β-sheet, the point of initial insertion of the RSL into the A β-sheet); the shutter (at top of the A β-sheet, the point of initial insertion of the RSL into the A β-sheet); and the gate (see, e.g., summary in Irving et al. (2000). Inhibitory serpins possess a high degree of conservation at many key amino acid residues located in the above regions.

In preferred embodiments of the invention a serpin, e.g., ACT, is conjugated to a proteinase such as PSA. ACT is readily available. It is commercially available (e.g, Scipac Ltd, Kent, United Kingdom) or it can be purified, for example from blood plasma or other sources, (see, e.g., Christensson et al., Eur. J. Biochem. 194:755-63, 1990). ACT can also be recombinantly produced using procedures that are routine in the art. For purposes such as recombinant production of ACT or related serpins, polypeptide and nucleic acid sequences are readily available in the art (see, e.g., U.S. Pat. No. 5,079,336). Exemplary human ACT protein sequences (unprocessed precursor) are provided in UniProt accession no. P01011 and NCBI accession no. NP_{—}001076. The mature protein is from amino acid 26-423 of UniProt accession P01011 (shown in SEQ ID NO:2). An exemplary mRNA sequence is provided in accession no. NM_{—}001085.

It is understood by one of skill that suitable ACT sequences can include variants that conserve the overall structure of the serpin. Such variants can be designed based on the structural analyses available in the art (e.g., references, supra). For example, variant ACT proteins may have residues introduced to facilitate linkage to PSA. Such proteins typically have at least 65% identity, more often at least 70%, 75%, 80%, 85%, 90%, 95%, 96%, 97%, 98%, or 99% sequence identity to the mature ACT and preserve the serpin structure.

### Serine Proteases

Serine proteases fall into two classes: the chymotrypsin family, which includes mammalian enzymes such as chymotrypsin, trypsin, elastase or kallikrein, and the substilisin family, which includes bacterial enzymes such as subtilisin. The two families share the same active site geometry and catalysis occurs via the same mechanism.

The active site of serine proteases is shaped as a cleft where the polypeptide substrate binds. Three residues that form the catalytic triad are important in the catalytic process. These are His 57, Asp 102 and Ser 195. The numbering is relative to chymotrypsinogen. During catalysis, an acyl enzyme intermediate between the substrate and the active site serine. During the second step, the acyl-enzyme intermediate is hydrolyzed by a water molecule to release the peptide and to restore the Ser-hydroxyl of the enzyme.

Many serine proteases are known in the art. For example, Rawlings and Barrett Rawlings (Meth. Enzymol. 244:19-61, 1994) have proposed a classification of proteases in families and clans. Families group sequences according to the alignment score of their catalytic domains. According to this classification, there are five clans and thirty families. Global classifications of proteases are accessible in the Merops and ExPASy websites.

In typical embodiments, the serine protease is a chymotrypsin-like protease, for example, PSA or trypsin. Often, the serine protease is PSA. PSA is a member of the glandular kallikrein gene family. Its substrates include semenogelin I and II, insulin-like growth factor binding protein 3, fibronectin, and laminin. Other related proteases can also be employed in the invention. For example, PSA, glandular kallikrein 2 (hK2), and tissue kallikrein (hK1) are members of the human glandular kallikrein family that are structurally similar. The mature forms of PSA and human glandular kallikrein 2 (hK2), which is also produced by the prostate gland, are 237-amino acid monomeric proteins that have 79% amino acid sequence identity.

In other embodiments, the protease is a trypsin or trypsin-related protein.

As noted above, serine proteases are well known in the art and can be readily obtained by purification or by expressing the protein using an expression vector. For example, PSA can be purified from a naturally occurring source, e.g., human seminal plasma, or can be produced recombinantly. PSA purification procedures are known (see, e.g., Sensabaugh & Blake, J. Urol. 144:1523-1526, 1990, Christenssen et al, supra). PSA is also available commercially (e.g., BioProcessing, Inc. Portland, Me.). Alternatively, PSA can be produced recombinantly using basic expression techniques.

For recombinant expression, exemplary PSA polypeptide sequences are available under UniProt accession no. P07288 and NCBI accession no. A32297. Exemplary nucleic acid sequences are provided in GenBank accession nos. AF335478, NM_{—}001030050, NM_{—}001030049, NM_{—}001030048, NM_{—}001030047, and NM_{—}001648. The sequence of human PSA precursor protein is provided, for example, in UniProt accession no. P07288. The mature form of PSA corresponds to residues 25-261. This exemplary protein sequence is provided in SEQ ID NO:1.

In some embodiments, PSA proteins, e.g., recombinantly expressed PSA proteins, with amino acid sequence changes can be employed in the inventions. For example, variant PSA proteins may have residues introduced to facilitate linkage to ACT. Such proteins typically have at least 65% identity, more often at least 70%, 75%, 80%, 85%, 90%, 95%, 96%, 97%, 98%, or 99% sequence identity to the mature PSA sequence (e.g., SEQ ID NO:1) and are recognized by antibodies to PSA, typically to the same extent as native PSA, such that a PSA-ACT complex made with a variant can, for example, serve as a control. Changes in amino acid sequence can be designed for example, based on the known structure of PSA.

In some embodiments, the invention provides a PSA-serpin, e.g., PSA-ACT or PSA-AT; or trypsin-anti-trypsin conjugates.

The terms “identical” or percent “identity,” in the context of two or more polypeptide sequences, refer to two or more sequences or subsequences that are the same or have a specified percentage of amino acid residues when compared and aligned for maximum correspondence over a comparison window or designated region) as measured using a BLAST or BLAST 2.0 sequence comparison algorithms with default parameters described below, or by manual alignment and visual inspection. The definition also includes sequences that have deletions and/or additions, as well as those that have substitutions. As described below, the preferred algorithms can account for gaps and the like. Preferably, identity exists over a region that is at least about 25 contiguous amino acids in length, or more preferably over a region that is 50-100 contiguous amino acids or 200, 300, or 400 or more contiguous amino acids.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

Methods of alignment of sequences for comparison are well-known in the art. Optimal alignment of sequences for comparison can be conducted, e.g., by the local alignment algorithm of Smith & Waterman, Adv. Appl. Math. 2:482 (1981), by the global alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Another example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, typically with the default parameters, to determine percent sequence identity for the nucleic acids and/or proteins used in the invention. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915)). For the purposes of this invention, the BLAST2.0 algorithm is used with the default parameters.

### Conjugation of a Serine Protease to a Serpin

The serine protease is often conjugated to the serpin using a chemical reaction. In this discussion, PSA is used as a representative serine protease and ACT is used as a representative serpin. It is understood that these techniques can be employed to covalently link any serine protease to a serpin.

In the context of this invention a “synthetic covalent linkage” refers to a covalent bond between two moieties that is not naturally occurring, but is generated by a chemical synthesis that is a purposeful execution of chemical reactions in order to get a product.

The terms term “stable covalent linkage” in the context of this application refers to a covalent linkage that has a property of being resistant to hydrolysis at a pH of about 7.0.

“Resistant to hydrolysis” in the context of this invention means that a serine proteinase-serpin conjugate, e.g., PSA-ACT, does not show appreciable hydrolysis. “No appreciable hydrolysis” or “no appreciable detection of freed PSA” is when less than about 10% of the PSA in a PSA-ACT complex is freed from the complex after at least 5 days, typically after at least 10 days or after at least 20 days, or after at least 30 days, at 2-8° C. in a serum, plasma, protein, or buffer solution that is at a pH of about 7.0.

As understood by one in the art, a stable proteinase-serpin, e.g., PSA-ACT, complex of the invention that is characterized by its resistance to hydrolysis at pH 7.0 is also resistant to hydrolysis at a lower pH and may be used at a pH other than about pH 7.0. For example, a stable covalently linked PSA-ACT complex of the invention that has a synthetic covalent bond may be employed in an assay performed at a pH of about 5.5, or about 6.0, or about 6.5. The complexes of the invention are also typically more stable at lower pH's, e.g., a pH of about 5.0 to about 6.5, than are spontaneously occurring PSA-ACT complexes that are not linked by a synthetic covalent bond.

PSA can be conjugated to a serpin, e.g., ACT, using many known methods, including chemical linkage and recombinant linkage. For example, PSA contains a variety of functional groups; e.g., carboxylic acid (COOH), free amine (—NH2) or sulfhydryl (—SH) groups, which are available for reaction with a suitable functional group on the serpin (and vice versa). PSA and/or a serpin can also be derivatized to expose or to attach additional reactive functional groups. The derivatization may involve attachment of any of a number of linker molecules, such as those available from Pierce Chemical Company, Rockford Ill.

There are many chemical means of joining a serpin and the PSA protein. Such methods are described, e.g., in Bioconjugate Techniques, Hermanson, Ed., Academic Press (1996). For example, a heterobifunctional coupling reagent can be used. The linking group can be a chemical crosslinking agent, including, for example, succinimidyl-(N-maleimidomethyl)-cyclohexane-1-carboxylate (SMCC). The linking group can also be an additional amino acid sequence(s), including, for example, a polyalanine, polyglycine or similarly, linking group.

Other chemical linkers include carbohydrate linkers, lipid linkers, fatty acid linkers, polyether linkers, e.g., PEG, etc. For example, poly(ethylene glycol) linkers are available from Shearwater Polymers, Inc. Huntsville, Ala. These linkers optionally have amide linkages, sulfhydryl linkages, or heterofunctional linkages.

A linker reagent can have a reactive nucleophilic functional group that is reactive with an electrophile present on a protein, e.g., PSA and/or ACT, to form a stable covalent bond. Useful electrophilic groups on a protein include, but are not limited to, aldehyde and ketone carbonyl groups. Useful nucleophilic groups on a linker include, but are not limited to, hydrazide, oxime, amino, hydrazine, thiosemicarbazone, hydrazine carboxylate, and arylhydrazide.

Carboxylic acid functional groups and chloroformate functional groups are also useful reactive sites for a linker because they can react with secondary amino groups of a protein to form an amide linkage. Also useful as a reactive site is a carbonate functional group on a linker, such as but not limited to p-nitrophenyl carbonate, which can react with an amino group of a protein, such as but not limited to N-methyl valine, to form a carbamate linkage.

In some embodiments, the PSA or serpin, e.g., ACT, can be modified at a lysine residue to introduce a sulfhydryl group. Reagents that can be used to modify lysines include, but are not limited to, N-succinimidyl S-acetylthioacetate (SATA) and 2-Iminothiolane hydrochloride (Traut's Reagent).

In another embodiment, the PSA or ACT can be modified at one or more carbohydrate groups to introduce a sulfhydryl group.

In another embodiment, the PSA or ACT can have one or more carbohydrate groups that can be oxidized to provide an aldehyde (—CHO) group (see for example, Laguzza, et al (1989) J. Med. Chem. 32(3):548-55). in Coligan et al, “Current Protocols in Protein Science”, vol. 2, John Wiley & Sons (2002), incorporated herein by reference.

The PSA-serpin conjugates of the invention can be prepared using various cross-linking reagents, including, but not limited to, reagents such as: BMPEO, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate), and including bis-maleimide reagents: DTME, BMB, BMDB, BMH, BMOE, BM(PEO)₃, and BM(PEO)₄, which are commercially available from Pierce Biotechnology, Inc. Rockford, Ill.). Bis-maleimide reagents allow the attachment of the thiol group of a cysteine residue of a protein to a thiol-containing protein moiety or linker intermediate, in a sequential or concurrent fashion. Other functional groups besides maleimide that are reactive with a thiol group of a protein or linker intermediate include iodoacetamide, bromoacetamide, vinyl pyridine, disulfide, pyridyl disulfide, isocyanate, and isothiocyanate.

PSA-serpin conjugates can also be made using a variety of bi-functional protein-coupling agents, such as N-succinimidyl-3-(2-pyridyldithiol) propionate (SPDP), iminothiolane (IT), bi-functional derivatives of imidoesters (such as dimethyl adipimidate HCl), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene).

In one embodiment, PSA is coupled to a serpin, e.g, ACT, using a maleimide coupling reagent. In such reactions, the carboxylic acids of water-soluble biopolymers such as proteins can be coupled to hydrazines, hydroxylamines and amines in aqueous solution using water-soluble carbodiimides such as 1-ethyl-3-(3-dimethylaminopropyl)carbodiimide (EDAC). Including N-hydroxysulfosuccinimide in the reaction mixture has been shown to improve the coupling efficiency of EDAC-mediated protein-carboxylic acid conjugations. To reduce intra- and interprotein coupling to lysine residues, which is a common side reaction, carbodiimide-mediated coupling is typically performed in a concentrated protein solution at a low pH, using a large excess of the nucleophile. The reaction creates an amide bond, which is extremely stable to hydrolysis and can only be hydrolysed in boiling alkali and under extremely acidic conditions. The covalent bond that is formed from this reaction is a peptide bond and therefore is as stable as any of the naturally occurring peptide bonds in the rest of the protein.

The serine protease can be linked to the serpin, e.g, PSA linked to ACT, at any site so long as the PSA is accessible for measurement, e.g., can be detected by an antibody used in a detection assay. Typically, linkage is not through the serine group on the protease that is site recognized by the anti-protease antibody used for detection of the protease (ie. Anti-PSA antibody) that is used in the detection assay. In some embodiments, the protease and serpin, e.g., PSA and ACT, are linked end to end, e.g., in a recombinant fusion protein. The orientation of the protease to the serpin does not matter, i.e., the N-terminus of the protease can be linked to the C-terminus of the serpin or the C-terminus of the protease can be linked to the N-terminus of the serpin.

### General Recombinant DNA Methods

This invention may employ routine techniques in the field of recombinant genetics for the preparation of serpin polypeptides, serine protease polypeptides, and/or serine protease-serpin fusion polypeptides. Basic texts disclosing the general methods of use in this invention include Sambrook & Russell, Molecular Cloning, A Laboratory Manual (3rd Ed, 2001); Kriegler, Gene Transfer and Expression: A Laboratory Manual (1990); and Current Protocols in Molecular Biology (Ausubel et al., eds., 1994-1999).

### Expression of PSA, Serpins and PSA-Serpin Conjugates

A serine protease, e.g., PSA, or serpin, e.g., ACT, or a fusion protein, e.g., comprising PSA-ACT, can be expressed using techniques well known in the art. Eukaryotic and prokaryotic host cells may be used such as animal cells, insect cells, bacteria, fungi, and yeasts. Methods for the use of host cells in expressing isolated nucleic acids are well known to those of skill and may be found, for example, in the general reference, supra. Accordingly, this invention also provides for host cells and expression vectors comprising the nucleic acid sequences described herein.

Nucleic acids encoding a serine protease, serpin, or a fusion protein can be made using standard recombinant or synthetic techniques. Nucleic acids may be RNA, DNA, or hybrids thereof. One of skill can construct a variety of clones containing functionally equivalent nucleic acids, such as nucleic acids that encode the same polypeptide. Cloning methodologies to accomplish these ends, and sequencing methods to verify the sequence of nucleic acids are well known in the art.

In some embodiments, the nucleic acids are synthesized in vitro. Deoxynucleotides may be synthesized chemically according to the solid phase phosphoramidite triester method described by Beaucage & Caruthers, Tetrahedron Letts. 22(20):1859-1862 (1981), using an automated synthesizer, e.g., as described in Needham-VanDevanter, et al., Nucleic Acids Res. 12:6159-6168 (1984). In other embodiments, the nucleic acids encoding the desired protein may be obtained by an amplification reaction, e.g., PCR.

One of skill will recognize many other ways of generating alterations or variants of a given polypeptide sequence. Most commonly, polypeptide sequences are altered by changing the corresponding nucleic acid sequence and expressing the polypeptide.

One of skill can select a desired nucleic acid or polypeptide of the invention based upon the sequences referred to herein and the knowledge readily available in the art regarding PSA and serpin structure and function. The physical characteristics and general properties of these proteins are known to skilled practitioners, including the active sites, as previously noted.

To obtain high level expression of a PSA, serpin, or PSA-serpin fusion, an expression vector is constructed that includes such elements as a promoter to direct transcription, a transcription/translation terminator, a ribosome binding site for translational initiation, and the like. Suitable bacterial promoters are well known in the art and described, e.g., in the references providing expression cloning methods and protocols cited hereinabove. Bacterial expression systems for expressing ribonuclease are available in, e.g., E. coli, Bacillus sp., and Salmonella (see, also, Palva, et al., Gene 22:229-235 (1983); Mosbach, et al., Nature 302:543-545 (1983). Kits for such expression systems are commercially available. Eukaryotic expression systems for mammalian cells, yeast, and insect cells are well known in the art and are also commercially available.

In addition to the promoter, the expression vector typically contains a transcription unit or expression cassette that contains all the additional elements required for expression of the nucleic acid in host cells. A typical expression cassette thus contains a promoter operably linked to the nucleic acid sequence encoding the PSA, or serpin, or fusion protein, and signals required for efficient polyadenylation of the transcript, ribosome binding sites, and translation termination. Depending on the expression system, the nucleic acid sequence encoding the PSA, serpin, or fusion protein, may be linked to a cleavable signal peptide sequence to promote secretion of the encoded protein by the transformed cell.

As noted above, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET15b, pET23D, pET-22b(+), and fusion expression systems such as GST and LacZ. Epitope tags can also be added to recombinant proteins to provide convenient methods of isolation, e.g., 6-his. These vectors comprise, in addition to the expression cassette containing the coding sequence, the T7 promoter, transcription initiator and terminator, the pBR322 ori site, a bla coding sequence and a lac1 operator.

The vectors comprising the nucleic acid sequences encoding the RNAse molecules or the fusion proteins may be expressed in a variety of host cells, including E. coli, other bacterial hosts, yeast, and various higher eukaryotic cells such as the COS, CHO and HeLa cells lines and myeloma cell lines. In addition to cells, vectors may be expressed by transgenic animals, preferably sheep, goats and cattle. Typically, in this expression system, the recombinant protein is expressed in the transgenic animal's milk.

The expression vectors or plasmids of the invention can be transferred into the chosen host cell by well-known methods such as calcium chloride transformation for E. coli and calcium phosphate treatment, liposomal fusion or electroporation for mammalian cells. Cells transformed by the plasmids can be selected by resistance to antibiotics conferred by genes contained on the plasmids, such as the amp, gpt, neo and hyg genes.

Once expressed, the expressed protein can be purified according to standard procedures of the art, including ammonium sulfate precipitation, column chromatography (including affinity chromatography), gel electrophoresis and the like (see, generally, R. Scopes, Protein Purification, Springer—Verlag, N.Y. (1982), Deutscher, Methods in Enzymology Vol. 182: Guide to Protein Purification, Academic Press, Inc. N.Y. (1990); Sambrook and Ausubel, both supra.

### Stability Assays

The PSA-serpin complexes of the invention are stable compared to prior art PSA-serpin complexes in that the covalent bond is pH-stable and irreversible under the following conditions. Prior art PSA-serpin, e.g., PSA-ACT complexes that occur spontaneously are subject to hydrolysis at pH of 7.0 or greater when stored for a period of time in the absence of excess purified serpin, e.g., ACT. To determine whether a PSA-ACT complex (or other PSA-serpin complex) of the invention is stable within the context of this invention, one of skill can incubate the PSA-serpin in a buffer, e.g., an albumin (either HSA or BSA or combination) buffer, at a pH of about 7.0. An exemplary buffer is a buffered protein solution that contains EDTA, sodium bicarbonate, phosphate and saline with 4 g/dL of protein. The PSA-serpin is maintained for a period of time, e.g., at least 5 days, or more often 30 or 36 days, open vial (see, Example 2). A complex is considered stable if there is no appreciable detection of PSA that is freed from the complex, e.g., if less than about 10% of the PSA in the complex is freed from the complex. Total PSA and free PSA can be detected, e.g., using immunological testing (e.g., Roche Elecsys kits for free and total PSA).

As understood in the art, the stable serpin-proteinase complexes, e.g., PSA-ACT complexes, of the invention may be employed at a pH other than 7.0. For example, a PSA-ACT complex that is linked by a synthetic covalent linkages, is also stable at or above a pH of about 5.5, e.g., about 6.0, about 6.5, about 7.0, or about 7.5 or above, and accordingly can be used in assays that are performed at those pHs. Stable PSA-ACT complexes having a synthetic covalent bond are also typically more stable at lower pH's in comparison to spontaneously forming PSA-ACT complexes.

### Kits

The invention also provides kits comprising the serine protease-serpin complex, e.g., PSA-ACT, of the invention. The PSA-ACT complex can be included as a control, e.g., in a kit that measures PSA levels in patients, including in multianalyte kits. Additionally, this invention can be applied to any immunoassay kit or control that contains proteases that through interaction with itself, or with other molecules impairs its own or other analyte's stability or performance.

### EXAMPLES

### Example 1

### Preparation of ACT-PSA Conjugate

ACT was chemically conjugated to PSA using a two step carbodiimide-mediated coupling reaction, which reaction is well known in the art. Briefly, PSA is prepared in a refrigerated phosphate buffered saline (PBS) solution at pH 6.0 with a concentration of 0.03 ng/mL. A solution of 1-Ethyl-3 (3-Dimethylaminopropyl Carbodiimide) HCl (EDAC) and N-Hydroxysulfosuccinamide (NHSS) is prepared and refrigerated. These two solutions are then reacted together and incubated for 2 hours. A solution of ACT is then prepared & added to the PSA/EDAC/NHSS solution. This mixture is allowed to incubate for 2 hours to allow for conjugation of the PSA with the ACT. Following the incubation, the conjugated PSA solution isn dialyzed to remove any unbound EDAC/NHSS using a membrane of a nominal molecular weight (MW) cutoff of 3500, against a refrigerated phosphate buffered saline solution at pH 7.0.

### Example 2

### Stability of an ACT-PSA Conjugate

Stability of the ACT-PSA conjugate generated in Example 1 was tested to determine of the conjugate was stable at a pH of above 7.0. Stability was tested in an open vial stability study and in accelerated stability studies. In an exemplary open vial stability evaluation, vials are refrigerated at 2°-8° C. for the time allotted and each working day are removed from the refrigerator and placed on the bench until reaching room temperature. The vials are then mixed and the caps are removed to allow air to enter. The caps are replaced and the samples are returned to the refrigerator. This assay is designed to mimic typical daily use of the product. In an accelerated stability study, vials are placed at various temperatures that are higher than their storage temperature in order to accelerate the analyte degradation.

The PSA-ACT conjugate is stable in serum, in buffer and in both a liquid and lyophilized state. For example, in a stability assay, it was observed that in serum, there was less than 2% change in recovery of total PSA and less than 3% change for free PSA recovery after 17 days of letting the vial come to room temperature, opening the vial, closing the vial and then storing it again at 2°-8° C. This was at a concentration of PSA of 33 ng/mL.

In buffer in an exemplary stability assay, there was a slight decrease after 36 days of less than 1% for free PSA and of about 1% for total PSA for the same protocol where the vials were stored at 2°-8° C., then removed to let them come to room temp, opening the vials, and then closing and returning the vials to 2°-8° C. The concentration of total PSA in this example was 40 ng/mL. Free PSA and Total PSA are stable at 0.1 ng/mL to 35 ng/mL.

### Example 3

### Use of ACT-PSA Conjugate

Stabilized PSA via the attachment of a serpin can be used for clinical laboratory controls, calibrators or reagents. An example of use for the stabilized PSA can be demonstrated in a multi-analyte control sample. Stabilizing the PSA via the conjugation to ACT allows for improved stability at various pH settings. Other clinical analytes of interest can be included in the multi-analyte control sample at the optimal pH for stability of the other clinical analytes in question. Un-conjugated PSA is relatively stable within only a narrow, slightly acid pH range, such as pH 5 to 6. This invention allows for PSA stability at a pH range of, e.g., above about pH 7 and is typically utilized between the pH range of 4 to 8. By allowing the pH of the control sample to be neutral to slightly basic, in order to provide stability of the other analytes in question, this invention allows for the inclusion of stable PSA into the control at normally unfavorable pH values for PSA e.g. neutral to basic pH.

For a demonstration of the effectiveness of the conjugates, comparison samples were prepared with un-conjugated PSA and conjugated PSA-ACT according to the described procedure. These samples were then subjected to open vial stability testing for 33 days. The analytes of interest were Total PSA & Free PSA and were tested on the Abbott Axsym instrument system. The results for the un-conjugated PSA sample yielded percent losses for Total PSA & Free PSA of 26.4% and 43.7%, respectively. The conjugated PSA-ACT sample yielded percent losses for Total PSA & Free PSA of 3.2% and 3.7%, respectively. This represents a significant improvement in the effectiveness of PSA stability for use in clinical laboratory controls, calibrators or reagents.

All publications, patents, accession number, and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

SEQ ID NO: 1

exemplary human PSA protein sequence (mature
protein)
IVGGWECEKHSQPWQVLVASRGRAVCGGVLVHPQWVLTAAHCIRNKSVIL
LGRHSLFHPEDTGQVFQVSHSFPHPLYDMSLLKNRFLRPGDDSSHDLMLL
RLSEPAELTDAVKVMDLPTQEPALGTTCYASGWGSIEPEEFLTPKKLQCV
DLHVISNDVCAQVHPQKVTKFMLCAGRWTGGKSTCSGDSGGPLVCNGVLQ
GITSWGSEPCALPERPSLYTKVVHYRKWIKDTIVANP

SEQ ID NO: 2

Exemplary human ACT protein sequence (mature
protein)
NSPLDEENLTQENQDRGTHVDLGLASANVDFAFSLYKQLVLKAPDKNVIF
SPLSISTALAFLSLGAHNTTLTEILKGLKFNLTETSEAEIHQSFQHLLRT
LNQSSDELQLSMGNAMFVKEQLSLLDRFTEDAKRLYGSEAFATDFQDSAA
AKKLINDYVKNGTRGKITDLIKDLDSQTMMVLVNYIFFKAKWEMPFDPQD
THQSRFYLSKKKWVMVPMMSLHHLTIPYFRDEELSCTVVELKYTGNASAL
FILPDQDKMEEVEAMLLPETLKRWRDSLEFREIGELYLPKFSISRDYNLN
DILLQLGIEEAFTSKADLSGITGARNLAVSQVVHKAVLDVFEEGTEASAA
TAVKITLLSALVETRTIVRFNRPFLMIIVPTDTQNIFFMSKVTNPKQA

What is claimed is:

1. A conjugate comprising a prostate specific antigen (PSA) linked to a serpin, wherein the PSA and serpin are linked by at least one synthetic covalent linkage.

2. The conjugate of claim 1, wherein the serpin is α1-antichymotrypsin (ACT).

3. The conjugate of claim 1, wherein the serine protease and the serpin are linked by an amide bond.

4. The conjugate of claim 1, wherein the serine protease and the serpin are linked by an amine-amine acid linkage.

5. The conjugate of claim 1, wherein the serine protease and the serpin are linked by a sulfhydryl-amine linkage.

6. The conjugate of claim 1, wherein the serine protease and the serpin are linked by a sulfhydryl linkage.

7. A conjugate comprising a prostate specific antigen (PSA) linked to a serpin, wherein the PSA and serpin are linked recombinantly to form a recombinant fusion protein.

8. The conjugate of claim 7, wherein the serpin is ACT.

9. A method of coupling a prostate specific antigen (PSA) to a serpin, the method comprising chemically linking the PSA to the serpin using a cross-linking agent.

10. The method of claim 9, wherein the serpin is ACT.

11. The method of claim 9, wherein the cross linking agent is a maleimide crossing linking reagent.

12. A method of coupling a protease to a serpin, the method comprising recombinantly expression a protease-serpin fusion protein.

13. The method of claim 12, wherein the protease is PSA and the serpin is ACT.

14. A composition comprising a conjugate of claim 1.

15. A kit comprising a conjugate of claim 1.

16. A composition comprising a conjugate of claim 2.

17. A kit comprising a conjugate of claim 2.
